(19) United States
(12) Patent Application Publication
(10) Pub. No.: US 2009/0234151 A1
(43) Pub. Date: Sep. 17, 2009

(54) Preparation of Mono-/Difluorinated Hydrocarbon Compounds

(75) Inventors: Laurent Saint-Jalmes, Vourles (FR); Daniel Uguen, Strasbourg (FR)

Correspondence Address: BUCHANAN, INGERSOLL & ROONEY PC, POST OFFICE BOX 1404, ALEXANDRIA, VA, 22313-1404, US

(73) Assignees: Rhodia Chimie, Aubervilliers (FR); CNRS, Paris, Cedex (FR)

(21) Appl. No.: 11/989,143

(22) PCT Filed: Jul. 7, 2006
(86) PCT No.: PCT/FR2006/001648

§ 371 Date: Jan. 16, 2009

(30) Foreign Application Priority Data
Jul. 19, 2005 (FR) 0507651

(51) Int. Cl. C07C 67/287; C07C 17/16; C07C 17/18

(52) U.S. Cl. 560/103; 570/141

(57) ABSTRACT

Mono- or difluorinated hydrocarbon compounds are prepared from an alcohol or a carbonylated compound by reacting one of these with a fluorinating reagent, optionally in the presence of a base, the fluorinating agent comprising a pyridinium reactant having the following formula (F), wherein R₀ is an alkyl or cycloalkyl radical:

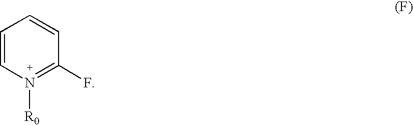

The subject of the present invention is a method for preparing monofluoro or difluoro hydrocarbon-based compounds.

Fluoro compounds are in general difficult to attain. The reactivity of the fluorine is such that it is difficult or even impossible to directly obtain fluoro derivatives.

One of the most used techniques for manufacturing the fluoro derivative consists in reacting a halo, generally chloro, derivative to exchange the halogen with a mineral fluorine, in general a fluoride of an alkaline metal, usually of high atomic weight.

Generally, the fluoride used is potassium fluoride which constitutes a satisfactory economic compromise.

Under these conditions, many methods, such as for example those described in FR 2 353 516 and in the article Chem. Ind. (1978) —56 have been described and used industrially to obtain aryl fluorides, aryls onto which electron-withdrawing groups are grafted.

Except in the case where the substrate is particularly suitable for this type of synthesis, this technique has drawbacks, of which the main ones are those which will be analyzed hereinbelow.

The reaction requires reagents like alkaline metal fluorides such as potassium fluoride, which are made relatively expensive by the specifications which they must meet in order to be suitable for this type of synthesis; they must be very pure, dry and in a suitable physical form.

Use is also made of reagents such as hydrofluoric acid which is liquid or diluted by dipolar aprotic solvents. However, hydrofluoric acid is too powerful a reagent and often results in undesired polymerization reactions or in tars.

In this case, and especially in the case where it is desired to have fluoro derivatives on a carbon of alkyl (including aralkyl) type that is electron poor due to the presence of electron-withdrawing type groups, a person skilled in the art finds himself faced with an alternative of which the terms are hardly encouraging; either very harsh conditions are chosen and mostly tars are obtained, or else it takes place under mild reaction conditions and, in the best of cases, the substrate is found unchanged. Finally, it should be mentioned that certain authors have proposed to carry out exchanges using, as a reagent, hydrofluoric acid salts in the presence of heavy elements in the form of oxides or fluorides. Among the elements used, mention should be made of antimony and the heavy metals such as silver or mercury.

It is important to find mild fluorination conditions, in particular that make it possible to convert the carbon-oxygen bonds to a carbon-fluorine bond.

Fluorinating reagents that enable this type of reaction to take place have already been proposed.

It is known to use an aminosulfur trifluoride (especially diethylaminosulfur trifluoride (DAST)) as a fluorinating agent (J. Org. Chem., 40, 3808 (1975); Tetrahedron, 44, 2875 (1988); J. Fluorine Chem., 43 (3), 405-13, (1989) and 42 (1), 137-43, (1989); EP 0 905 109). In particular, it makes it possible to convert a carbonyl group to a difluoromethylene group.

The disadvantage of DAST is in resulting in foul-smelling by-products, which are difficult to remove from the reaction medium.

H. Hayashi et al. have described 2,2-difluoro-1,3-dimethylimidazoline as a novel fluorinating agent that allows the conversion of alcohols to monofluoro compounds and of aldehydes/ketones to gem-difluoro compounds.

Said reagent does not seem very stable and the yields given are difficult to attain.

It was therefore desirable to provide an improved method making it possible to carry out the fluorination under better conditions.

A method has now been found, and it is this which constitutes the subject of the present invention, for preparing a monofluoro or difluoro hydrocarbon-based compound from an alcohol or from a carbonyl-based compound which comprises the reaction of one of them with a fluorinating reagent, optionally in the presence of a base, which is characterized in that the fluorinating agent is a reagent comprising a pyridinium unit corresponding to the following formula:

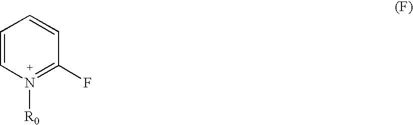

in said formula:
R₀ represents an alkyl or cycloalkyl group.

In the present text, the term “alkyl” is understood to mean a linear or branched hydrocarbon-based chain having from 1 to 6 carbon atoms and preferably from 1 to 4 carbon atoms.

Examples of preferred alkyl groups are, in particular, methyl, ethyl, propyl, isopropyl, butyl, isobutyl, or t-butyl groups.

The term “cycloalkyl” is understood to mean a cyclic or monocyclic hydrocarbon-based group comprising from 3 to 7 carbon atoms, preferably 5 or 6 carbon atoms.

It should be noted that the R₀ group could have another meaning, for example benzyl, but from an economic viewpoint, there is no advantage in having a complicated R₀ group. Thus, the C₁-C₄ alkyl groups, and more particularly the methyl group, are preferred.

According to the method of the invention, the fluorination is carried out using the reagent for fluorinating an alcohol or a carbonyl-based, aldehyde or ketone compound.

A first embodiment of the invention consists in preparing a monofluoro compound from a corresponding hydroxylated compound (alcohol).

Another variant of the invention consists in preparing a gem-difluoro compound from a carbonyl-based compound.

A fluorinating reagent comprising the unit corresponding to the formula (F) is involved in the method of the invention.

One preferred reagent is to make use of 1-alkyl- or 1-cycloalkyl-2-fluoropyridinium, but the invention also envisages the case where said unit is included in a polycyclic structure such that, for example, the pyridinium ring is fused to a saturated, unsaturated or aromatic ring having 5 or 6 carbon atoms.

As more specific examples, mention may be made of 1-alkyl- or 1-cycloalkyl-2-fluoroquinolinium.

The invention does not exclude the presence of one or more (to a maximum of 4) substituents on a or the rings of the reagent, in particular on the pyridinium ring.

As examples, given by way of illustration, mention may especially be made of alkyl or alkoxy groups having from 1 to 4 carbon atoms, a halogen atom (F, Cl, Br, I) or an electron-withdrawing group for example a nitro group or a carboxylate of an alkyl having from 1 to 4 atoms.

According to another embodiment of the invention, the fluoro reagent may be prepared in situ by using, combined with a fluoride source, a halogenated reagent comprising a pyridinium unit corresponding to the following formula:

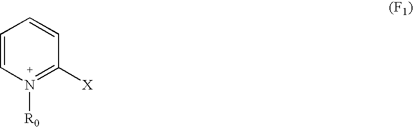

in said formula:
X represents a halogen atom with a higher ranking than fluorine, preferably chlorine, bromine, or iodine; and
R₀ represents an alkyl or cycloalkyl group.

It should be noted that in the pyridinium unit of formula (F) or (F₁), the nitrogen atom is quaternized. The counterion with which it is associated and which is symbolized by Y⁻ results from the method of preparing said unit. It is preferably a halide, or a sulfonate or carboxylate group.

As examples of halides, mention may be made of fluoride, chloride, bromide or iodide.

As for the sulfonate group, it may be represented by the formula RₐSO₃⁻ in which Rₐ is a hydrocarbon-based group.

In said formula, Rₐ is a hydrocarbon-based group of any nature. However, it is advantageous from an economic viewpoint that Rₐ is of a simple nature, and more particularly represents a linear or branched alkyl group having from 1 to 4 carbon atoms, preferably a methyl or ethyl group, but it may also represent for example a phenyl or tolyl group or a trifluoromethyl group. Among the RₐSO₃⁻ groups, the preferred group is a triflate group which corresponds to an Rₐ group representing a trifluoromethyl group.

Y⁻ may also be a carboxylate group which may be represented by the formula R_{b}CO₂⁻ in which R_{b} is a hydrocarbon-based group.

As for the sulfonate group, the nature of R_{b} is not very important but it is economically desirable that R_{b} be an alkyl group having from 1 to 4 carbon atoms, preferably a methyl group.

As fluorinating reagents preferably used in the method of the invention, mention may especially be made of:
2-fluoro-N-methylpyridinium tosylate;
2-fluoro-N-methylpyridinium triflate;
2-fluoro-N-methylpyridinium fluoride;
N-methyl-2-fluoroquinolinium triflate; and
N-methyl-2-fluoroquinolinium fluoride.

The amount of fluorinating reagent used is expressed relative to the amount of substrate, alcohol or carbonyl-based compound. It is preferably at least equal to the stoichiometric amount. It is such that the ratio between the number of moles of fluorinating reagent and the number of moles of substrate usually varies between 1 and 3 and is preferably between 1.5 and 2.

According to the method of the invention, an alcohol or a carbonyl-based compound is reacted with the fluorinating reagent of the invention, in the presence of a base and in an organic medium.

### Alcohol

As for the alcohol, it more particularly corresponds to the general formula (I):

R₁—OH (I)

in said formula (I):
R₁ represents a hydrocarbon-based group having from 1 to 30 carbon atoms, which may be a linear or branched, saturated or unsaturated acyclic aliphatic group; a saturated, unsaturated or aromatic cycloaliphatic group; a linear or branched, saturated or unsaturated aliphatic group bearing a cyclic substituent.

The alcohol which is involved in the method of the invention corresponds to the formula (I) in which R₁ represents a linear or branched, saturated or unsaturated acyclic aliphatic group.

More specifically, R₁ represents a linear or branched alkyl, alkenyl, alkadienyl or alkynyl group preferably having from 1 to 30 carbon atoms.

The hydrocarbon-based chain may possibly be:
interrupted by one of the following groups:
   —O—, —CO—, —COO—, —OCOO—, —S—, —SO₂—, —NR₂—, —CO—NR₂—,
in these formulae, R₂ represents hydrogen or an alkyl group, preferably a methyl or ethyl group; and/or
a bearer of one of the following substituents:
   —OH, —OCOO—, —COOR₂, —CHO, —NO₂, —X, —CF₃,
in these formulae, R₂ having the meaning given previously.

The linear or branched, saturated or unsaturated, acyclic aliphatic remainder may possibly bear a cyclic substituent. The term “ring” is understood to mean a saturated, unsaturated or aromatic carbocyclic or heterocyclic ring.

The acyclic aliphatic remainder may be linked to the ring by a valence bond or by one of the following groups:
—O—, —CO—, —COO—, —OCOO—, —S—, —SO₂—, —NR₂—, —CO—NR₂—,
in these formulae, R₂ having the meaning given previously.

As examples of cyclic substituents, it is possible to envisage cycloaliphatic, aromatic or heterocyclic substituents, especially cycloaliphatic substituents comprising 6 carbon atoms in the ring or benzene substituents.

In the general formula (I) of the alcohols, R₁ may also represent a carbocyclic group that is saturated or that comprises 1 or 2 unsaturations in the ring, generally having from 3 to 7 carbon atoms, preferably 6 carbon atoms in the ring.

As preferred examples of R₁ groups, mention may be made of cyclohexyl or cyclohexene/cyclohexenyl groups.

It should be noted that when the R₁ group represents a ring, the invention also includes the case where the ring may bear one or more substituents insofar as they do not interfere with the method of the invention. Mention may especially be made of alkyl or alkoxy groups having from 1 to 4 carbon atoms.

The method is easily carried out with most alcohols.

As more particular examples of alcohols, mention may be made of:
lower aliphatic alcohols having from 1 to 5 carbon atoms, such as for example, methanol, ethanol, trifluoroethanol, propanol, isopropyl alcohol, butanol, isobutyl alcohol, sec-butyl alcohol, tert-butyl alcohol, pentanol, isopentyl alcohol, sec-pentyl alcohol and tert-pentyl alcohol, ethylene glycol monoethyl ether, methyl lactate, isobutyl lactate, methyl D-lactate and isobutyl D-lactate;
higher aliphatic alcohols having at least 6 and up to around 20 carbon atoms, such as for example, hexanol, heptanol, isoheptyl alcohol, octanol, isooctyl alcohol, 2-ethylhexanol, sec-octyl alcohol, tert-octyl alcohol, nonanol, isononyl alcohol, decanol, dodecanol, tetradecanol, octadecanol, hexadecanol, oleyl alcohol, eicosyl alcohol, and diethylene glycol monoethyl ether;
cycloaliphatic alcohols having from 3 to about 20 carbon atoms, such as for example, cyclopropanol, cyclobutanol, cyclopentanol, cyclohexanol, cycloheptanol, cyclooctanol, cyclododecanol, tripropylcyclohexanol, methylcyclohexanol and methylcycloheptanol, cyclopentenol, cyclohexenol; and
an aliphatic alcohol bearing an aromatic group having from 7 to around 20 carbon atoms, such as for example, benzyl alcohol, phenethyl alcohol, phenylpropyl alcohol, phenyloctadecyl alcohol and naphthyldecyl alcohol.

It is also possible to use polyols, especially polyoxyethylene glycols, such as for example, ethylene glycol, diethylene glycol, triethylene glycol, propylene glycol and glycerol.

Among the aforementioned alcohols, the following are preferably used in the method of the invention: aliphatic or cycloaliphatic alcohols, preferably primary or secondary aliphatic alcohols having 1 to 4 carbon atoms.

One variant of the method of the invention consists in using a terpene alcohol and more particularly a terpene alcohol of formula (Ia):

T-OH (Ia)

in said formula (Ia):
T represents the remainder of a terpene alcohol having a number of carbon atoms which is a multiple of 5.

In the description which follows of the present invention, the term “terpene” is understood to mean the oligomers derived from isoprene.

More specifically, the alcohol used corresponds to the general formula (Ia) in which the remainder T represents a hydrocarbon-based group having from 5 to 40 carbon atoms and more particularly a linear or branched, saturated or unsaturated aliphatic group; a monocyclic or polycyclic, saturated, unsaturated or aromatic, cycloaliphatic group comprising rings having from 3 to 8 carbon atoms.

It will be specified, without however limiting the scope of the invention, that the remainder T represents the remainder of:
a linear or branched, saturated or unsaturated, aliphatic terpene alcohol;
a saturated or unsaturated, or aromatic, monocyclic, cycloaliphatic terpene alcohol;
a polycyclic, cycloaliphatic terpene alcohol comprising at least two saturated and/or unsaturated carbocycles.

Regarding the remainder T of a linear or branched, saturated or unsaturated, aliphatic terpene alcohol, the number of carbon atoms varies between 5 and 40 carbon atoms. As more specific examples of remainder T, mention may be made of the groups comprising 8 carbon atoms, that are saturated or that have a double bond, and that bear two methyl groups, preferably in position 3 and 7.

When this is a monocyclic compound, the number of carbon atoms in the ring may vary widely from 3 to 8 carbon atoms but it is preferably 5 or 6 carbon atoms.

The carbocycle may be saturated or comprising 1 or 2 unsaturations in the ring, preferably 1 to 2 double bonds which are usually in position α of the oxygen atom.

In the case of an aromatic terpene alcohol, the aromatic ring is generally a benzene ring.

The compound may also be polycyclic, preferably bicyclic, which means that at least two rings have two carbon atoms in common. In the case of polycyclic compounds, the number of carbon atoms in each ring varies between 3 and 6: the total number of carbon atoms being preferably equal to 7.

Given below are examples of a commonly encountered bicyclic structure:

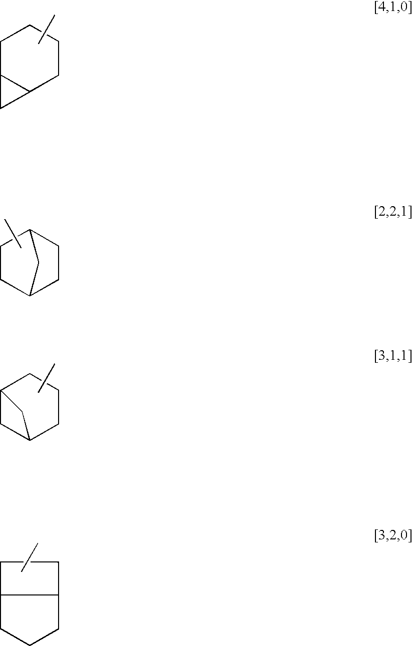

In the case of a ring, the presence of substituents is not excluded insofar as they are compatible with the envisaged application. The substituents usually borne by the carbocycle are one or more alkyl groups, preferably three methyl groups, a methylene group (corresponding to an exocyclic bond), an alkenyl group, preferably an isopropenyl group.

As examples of terpene alcohols capable of being used, mention may be made of:
saturated or unsaturated aliphatic terpene alcohols such as:
   3,7-dimethyloctanol;
   hydroxycitronellol;
   1-hydroxy-3,7-dimethyl-7-octene;
   nerol;
   geraniol;
   linalool; and
   citronellol;
aromatic cycloaliphatic terpene alcohols such as:
   thymol;
saturated or unsaturated, monocyclic or polycyclic, cycloaliphatic terpene alcohols such as:
   chrysanthemyl alcohol;
   1-hydroxyethyl-2,2,3-trimethylcyclopentane;
   β-terpineol;
   1-methyl-3-hydroxy-4-isopropylcyclohexene;
   α-terpineol;
   terpinene-4-ol;
   1,3,5-trimethyl-4-hydroxymethylcyclohexene; and
   isoborneol.

Among the aforementioned alcohols, the preferred alcohols are the following:
chrysanthemyl alcohol;
3,7-dimethyloctanol;
geraniol;
linalool;
citronellol;
hydroxycitronellol;
nerol;
thymol;
menthol; and
isoborneol.

### Carbonyl-Based Compound

Involved in the method of the invention, as substrates, may be an aldehyde or ketone (or diketone) corresponding to one of the general formulae:

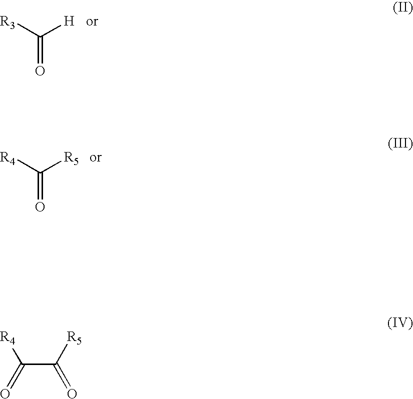

in said formulae:
R₃, R₄ and R₅, being identical or different, represent a hydrocarbon-based group comprising from 1 to 40 carbon atoms which may be a linear or branched, saturated or unsaturated acyclic aliphatic group; a monocyclic or polycyclic, saturated, unsaturated or aromatic carbocyclic or heterocyclic group; or a chaining of the aforementioned groups;
the R₄ and R₅ groups may be linked together to form a ring comprising 5 or 6 atoms; and
the R₄ and R₅ groups do not comprise hydrogen atoms on the carbon atom in position α with respect to the carbonyl group.

The invention may use symmetrical ketones or diketones if, in the formulae (III) or (IV), R₄ is identical to R₅ and dissymmetrical ketones or diketones if R₄ is different to R₅.

More specifically in the formulae (II) to (IV), R₃, R₄ and R₅ represent a hydrocarbon-based group having from 1 to 20 carbon atoms which may be a linear or branched, saturated or unsaturated acyclic aliphatic group; a monocyclic or polycyclic, saturated, unsaturated or aromatic carbocyclic or heterocyclic group; or a linear or branched, saturated or unsaturated, aliphatic group bearing a cyclic substituent.

R₃, R₄ and R₅ preferably represent a linear or branched, saturated acyclic aliphatic group preferably having from 1 to 12 carbon atoms, and even more preferably from 1 to 4 carbon atoms.

The invention does not exclude the presence of an unsaturation on the hydrocarbon-based chain such as one or more double bonds which may be conjugated or unconjugated, or a triple bond.

The hydrocarbon-based chain may optionally be interrupted by a heteroatom (for example, oxygen or sulfur) or by a functional group insofar as this does not react and in particular mention may be made of a group such as —CO— especially.

The hydrocarbon-based chain may optionally bear one or more substituents (for example, halogen, ester) insofar as they do not interfere with the ketonization reaction.

The linear or branched, saturated or unsaturated, acyclic aliphatic group may optionally bear a cyclic substituent. The term “ring” is understood to mean a saturated, unsaturated or aromatic carbocyclic or heterocyclic ring.

The acyclic aliphatic group may be connected to the ring by a valence bond, a hetero atom or a functional group such as an oxy, carbonyl, carboxy, sulfonyl, etc. group.

As examples of cyclic substituents, it is possible to envisage cycloaliphatic, aromatic or heterocyclic substituents, especially cycloaliphatic substituents comprising 6 carbon atoms in the ring or benzene substituents, these cyclic substituents themselves optionally bearing a substituent of any type insofar as they do not disturb the reactions taking place in the method of the invention. Mention may be made, in particular, of alkyl or alkoxy groups having from 1 to 4 carbon atoms.

Among the aliphatic groups bearing a cyclic substituent, cycloalkylalkyl groups, for example cyclohexylalkyl groups or aralkyl groups having from 7 to 12 carbon atoms, especially benzyl or phenylethyl groups, are more particularly targeted.

In the formulae (III) or (IV), R₃, R₄ and R₅ may also represent a saturated or unsaturated carbocyclic group preferably having 5 or 6 carbon atoms in the ring; a saturated or unsaturated heterocyclic group especially comprising 5 or 6 atoms in the ring, including 1 or 2 heteroatoms such as nitrogen, sulfur and oxygen atoms; a monocyclic, aromatic, carbocyclic or heterocyclic group, preferably a phenyl, pyridyl, pyrazolyl or imidazolyl group or a fused or unfused polycyclic group, preferably a naphthyl group.

Since one of the R₃, R₄ and R₅ groups comprises a ring, this may also be substituted. The nature of the substituent may be any insofar as it does not interfere with the main reaction. The number of substituents is generally at most 4 per ring but usually equal to 1 or 2.

Among all the meanings given previously, R₃ preferably represents a linear or branched alkyl group having from 1 to 12 carbon atoms, preferably from 1 to 6 carbon atoms or a phenyl group.

As mentioned previously, the R₄ and R₅ groups do not comprise hydrogen atoms on the carbon atom in position α with respect to the carbonyl group.

Thus, the carbon atoms in position α with respect to the carbonyl group are tertiary carbon atoms. An example of a tertiary carbon atom may be represented by the formula (R₆)(R₇)(R₈)C—in which R₆, R₇ and R₈ represent, in particular, a halogen atom, preferably a fluorine atom; a linear or branched alkyl group having from 1 to 6 carbon atoms; the R₆, R₇ and R₈ groups, which may also form a ring, for example a phenyl group optionally included in a polycyclic structure such as, for example, of naphthalenic type.

In the formulae (III) and (IV), the R₄ and R₅ groups may be bonded together to form a ring comprising 5 or 6 atoms: as the carbon atoms located at position α on both sides of the carbonyl group [formula (III)] or of the carbonyl groups [formula (IV)] are tertiary this means that they are either substituted (as mentioned above) or are included in an unsaturated or aromatic ring having 5 or 6 atoms, preferably a benzene ring.

As specific examples of ketones which may be used in the method of the invention, mention may more particularly be made of:
benzophenone;
2-methylbenzophenone;
2,4-dimethylbenzophenone;
4,4′-dimethylbenzophenone;
2,2′-dimethylbenzophenone;
4,4′-dimethoxybenzophenone;
4-benzoylbiphenyl;
fluorenone; and
phenanthrene-9,10-dione.

Given below are examples of alcohols and of carbonyl-based compounds used in the method of the invention: 1-decanol, 1-decanol, isopropyl mandelate, anisaldehyde, terephthaldehyde and phenanthrene-9,10-dione.

### Base

A base is optionally involved in the method of the invention, the role of which is to trap the leaving group which is an acid halide.

The characteristic of the base is that it has a pKa at least greater than or equal to 4, preferably between 5 and 14, and more preferably between 7 and 11.

The pKa is defined as the ionic dissociation constant of the acid/base pair, when water is used as a solvent.

For the choice of a base having a pKa as defined by the invention, reference may be made, amongst others, to the Handbook of Chemistry and Physics, 66th edition, p. D-161 and D-162.

Another requirement that governs the choice of the base is that it be non-nucleophilic, that is to say that it is not substituted for the substrate in the reaction.

Another characteristic of the base is that it is preferred that it be soluble in an organic medium.

Among the bases suitable for the method of the invention, mention may be made, amongst others, of mineral bases such as carbonates, hydrogencarbonates, phosphates, or hydrogenphosphates of alkaline metals, preferably of sodium, potassium or cesium or of alkaline-earth metals, preferably of calcium, barium or magnesium.

Also suitable are organic bases such as tertiary amines and mention may more particularly be made of triethylamine, tri-n-propylamine, tri-n-butylamine, methyldibutylamine, methyldicyclohexylamine, ethyldiisopropylamine, N,N-diethylcyclohexylamine, pyridine, dimethylamino-4-pyridine, N-methylpiperidine, N-ethylpiperidine, N-n-butylpiperidine, 1,2-dimethylpiperidine, N-methylpyrrolidine, 1,2-dimethylpyrrolidine.

Among the bases, preferably triethylamine is chosen.

The amount of base used expressed relative to the pyridinium salt is at least equal to the stoichiometric amount. More preferably it is such that the ratio between the number of moles of pyridinium salt and the number of moles of base preferably varies between 1 and 3 and even more preferably between 1.5 and 2.

### Fluoride Source

The fluoride is introduced into the medium in the form of salt(s).

Mention may be made, by way of example, of hydrofluoric acid; the salts such as for example potassium fluoride or ammonium fluoride.

It is also possible to make use of quaternary ammonium fluorides, preferably tetraalkylammonium fluorides, and more particularly tetrapropylammonium and tetrabutylammonium fluorides; tetraalkylammonium hydrogendifluorides, preferably ammonium hydrogendifluoride.

Preferably, tetrabutylammonium fluoride (TBAT) is chosen.

The amount of fluoride source used expressed relative to the oxygenated substrate is at least equal to the stoichiometric amount. More preferably, it is such that the ratio between the number of moles of fluoride and the number of moles of substrate (alcohol or ketone) preferably varies between 1 and 3, and even more preferably between 1.5 and 2.

### Organic Solvent

The reaction is generally carried out in the presence of a reaction solvent.

A solvent is chosen which is inert under the reaction conditions.

As more specific examples of solvents that are suitable for the present invention, mention may preferably be made of the polar aprotic solvents such as dimethyl sulfoxide, sulfolane or linear or cyclic carboxamides, such as N,N-dimethylacetamide (DMAC), N,N-diethylacetamide, dimethylformamide (DMF) or diethylformamide; aliphatic or aromatic nitriles, preferably acetonitrile, propionitrile, butanenitrile, isobutanenitrile, pentanenitrile, 2-methylglutaronitrile, adiponitrile, benzonitrile, tolunitrile, malonitrile, 1,4-benzonitrile.

As other examples of less polar organic solvents that are suitable for the invention, mention may especially be made of halogenated or nonhalogenated aliphatic, cycloaliphatic or aromatic hydrocarbons; or ethers.

It is also possible to make use of aliphatic and cycloaliphatic hydrocarbons, more particularly paraffins such as especially hexane, heptane, octane, isooctane, nonane, decane, undecane, tetradecane, petroleum ether and cyclohexane; aromatic hydrocarbons such as especially benzene, toluene, xylenes, ethylbenzene, diethylbenzenes, trimethylbenzenes, cumene, pseudocumene, and petroleum cuts composed of a mixture of alkylbenzenes, especially Solvesso® type cuts.

It is also possible to use aliphatic or aromatic halogenated hydrocarbons, mention may more particularly be made of the perchlorinated hydrocarbons such as, in particular, tetrachloroethylene and hexachloroethane; partially chlorinated hydrocarbons such as dichloromethane, chloroform, 1,2-dichloroethane, 1,1,1-trichloroethane, 1,1,2,2-tetrachloroethane, pentachloroethane, trichloroethylene, 1-chlorobutane, 1,2-dichlorobutane; monochlorobenzene, 1,2-dichloro-benzene, 1,3-dichlorobenzene, 1,4-dichlorobenzene, 1,2,4-trichlorobenzene or mixtures of various chlorobenzenes.

Preferably, dichloromethane or chloroform are chosen.

As examples of solvents, mention may be made of aliphatic, cycloaliphatic or aromatic ethers and, more particularly, diethyl ether, dipropyl ether, diisopropyl ether, dibutyl ether, methyl tert-butyl ether, dipentyl ether, diisopentyl ether, ethylene glycol dimethyl ether (or 1,2-dimethoxyethane), diethylene glycol dimethyl ether (or 1,5-dimethoxy-3-oxapentane), dioxane or tetrahydrofuran.

It is also possible to use a mixture of organic solvents.

The amount of organic solvent used is preferably chosen such that the weight concentration of the starting substrate in the solvent is between 5 and 40%, preferably between 10 and 20%.

The reaction is generally carried out at a temperature between 0° C. and 140° C., preferably between 80° C. and 100° C.

The fluorination reaction is generally carried out under atmospheric pressure but preferably under a controlled atmosphere of inert gases. It is possible to establish an atmosphere of rare gases, preferably argon but it is more economical to use nitrogen. A pressure slightly greater than or less than atmospheric pressure may be suitable.

From a practical point of view, the reaction is simple to implement.

The order the reagents are used in is not critical. One preferred variant consists in charging the substrate, the solvent and the fluorinating agent and then the base and heating to the desired temperature.

The reaction time is very variable. It may be from 1 to 24 hours and is preferably between 8 and 15 hours.

At the end of the reaction, the fluoro product is recovered by implementing the usual techniques of a person skilled in the art.

Generally, water is added to dissolve the salts in the aqueous phase and a non-miscible solvent, for example dichloroethane, toluene or monochlorobenzene is added in order to recover the fluoro compound obtained in the organic phase.

The aqueous and organic phases are then separated.

The fluoro compound is recovered according to conventional separation methods, for example by distillation or by crystallization in a suitable solvent, especially an ether such as isopropyl ether or else an alcohol such as methanol, ethanol or isopropanol.

The fluorinating reagents according to the invention comprising the units (F) or (F₁) may be prepared conventionally.

Reference may especially be made to the works by P. H. Gross et al. [J. Org. Chem. (1991), 56, 509-513) for preparing 2-fluoro-N-methylpyridinium tosylate and Marvell et al., J. Am. Chem. Soc. (1929), 51, 3640 for preparing 2-chloro-N-methylpyridinium tosylate.

One route for attaining said reagents consists in carrying out a reaction for alkylating a 2-halopyridine which may be represented by the following formula:

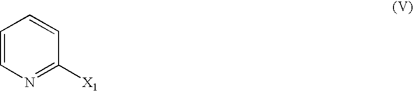

in said formula, X₁ represents a fluorine, chlorine, bromine or iodine atom.

As alkylating agents, use may be made of alkyl halides, preferably having a low C₁-C₄ carbon number and preferably methyl iodide or bromide.

It is also possible to use a sulfonic acid or carboxylic acid halide that may be represented by the following formulae:

RₐSO₃X₂ (VI) and

R_{b}CO₂X₂ (VII)

in which Rₐ and R_{b} have the meaning given previously and X₂ represents a halogen atom, chlorine, bromine or iodine.

The 2-halopyridine is reacted with an alkylating agent as mentioned above.

Generally, the alkylating agent is in a slight excess, the molar ratio between the alkylating agent and the 2-halopyridine advantageously varies between 1.1 and 1.2.

The temperature of the alkylation reaction is generally between 0° C. and 80° C., preferably between 20° C. and 50° C.

The reaction is carried out in the presence of an organic solvent that is inert under the reaction conditions.

As examples of solvents, mention may especially be made of halogenated or nonhalogenated aliphatic or aromatic hydrocarbons or else of nitrites. Reference may be made to the lists given previously in the present text.

Dichloromethane, chlorobenzene and toluene are preferred.

The pyridinium salt formed precipitates in the reaction medium.

The precipitate is recovered according to conventional solid/liquid separation techniques, preferably by filtration.

The precipitate may be washed, preferably using the organic solvent used during the reaction, then the solvent is removed by evaporation.

It is then used in the method of the invention.

According to one variant of the invention, it is possible to prepare the 1-alkyl- or 1-cycloalkyl-2-fluoropyridinium from a reagent comprising another halogen, for example a 1-alkyl- or 1-cycloalkyl-2-chloropyridinium by carrying out the exchange of chlorine with a fluorine atom, by using a fluoride of an alkaline metal, preferably of sodium or potassium.

The starting reagent is suspended in an organic solvent such as mentioned previously, for example acetonitrile, then the alkaline metal fluoride is added in powder form in an amount ranging from the stoichiometric amount up to an amount in excess, for example, of 20%.

The alkaline metal chloride formed is separated according to conventional solid/liquid separation techniques, preferably by filtration.

The fluoro reagent is then recovered.

Exemplary embodiments of the invention are given below by way of illustration and nonlimitingly.

The yield defined in the examples corresponds to the ratio between the number of moles of product formed and the number of moles of substrate used.

The examples A to K relate to the preparation of the fluorinating reagent and the following examples, to their use for preparing monofluoro compounds (Examples 1 to 5) or difluoro or polyfluoro compounds (Examples 6 to 8).

### EXAMPLES

### Example A

### Preparation of 2-chloro-N-methylpyridinium tosylate

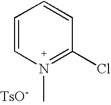

In a 25 ml round-bottomed flask topped with a condenser, 2-chloropyridine (2.3 g, 20.6 mmol) and methyl tosylate (3.83 g, 20.6 mmol) were heated at 80-85° C. for one hour.

Hot toluene (15 ml) was then added before the mixture was cooled and crystallized.

The whole mixture was left stirring for 10 minutes and the mixture was left to return to room temperature.

The crystallized bottom phase was recovered.

The product was in the form of a white solid and was obtained with a yield of 88% (5.4 g).

The NMR characteristics were the following:

¹H NMR (300 MHz, CDCl₃): 2.26 (s, 3H); 4.35 (s, 3H); 7.04 (d, J=8 Hz, 2H); 7.56 (d, J=8 Hz, 2H); 7.85-7.91 (m, 2H); 8.38-8.44 (m, 1H); 9.34 (d, J=5.3 Hz, 1H).

¹³C NMR (75 MHz, CDCl₃):
Primaries: 21.3; 47.8.
Secondaries: -
Tertiaries: 125.7; 126.7 (2C); 128.8 (2C); 129.5; 149.6; 154.5.
Quaternaries: 140.1; 143.4; 147.4.

### Example B

### Preparation of 2-chloro-N-methylpyridinium triflate

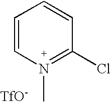

In a 25 ml round-bottomed flask, 2-chloropyridine (2 g, 20.6 mmol) was diluted in 15 ml of toluene.

Using a syringe, methyl triflate (2.33 ml, 20.6 mmol) was added to this solution.

The mixture was left stirring magnetically at room temperature for one hour.

The precipitate was then filtered over a Büchner funnel.

The traces of solvent were removed via evaporation under a reduced pressure of around 20 mmHg.

The product was in the form of a white solid and was obtained with a yield of 99%.

The NMR characteristics were the following:

¹H NMR (300 MHz, DMSO): 4.33 (s, 3H); 8.08 (ddd, J=7.6 Hz, J=6.2 Hz, J=1, 3 Hz, 1H); 8.37 (dd, J=8.3 Hz, J=1.3 Hz, 1H), 8.58 (ddd, J=8 Hz, J=8 Hz, J=1.6 Hz, 1H); 9.16 (dd, J=6.2 Hz, J=1.6 Hz, 1H).

¹³C NMR (75 MHz, DMSO):
Primaries: 47.3.
Secondaries: -
Tertiaries: 126.1; 129.4; 147.0; 148.2.
Quaternaries: 170.6 (q, J=322.2 Hz); 121.1 (q, J=322 Hz).

### Example C

### Preparation of 2-fluoro-N-methylpyridinium tosylate from 2-fluoropyridine

In a 25 ml round-bottomed flask topped with a condenser, 2-fluoropyridine (2 g, 20.6 mmol) was diluted in 15 ml of toluene.

Using a syringe, methyl tosylate (3.83 g, 20.6 mmol) was added to this solution.

The mixture was refluxed with magnetic stirring overnight.

During the reaction a second yellow phase appeared which crystallized at room temperature.

The precipitate was then filtered over a Büchner funnel.

The traces of solvent were removed via evaporation under a reduced pressure of around 20 mmHg.

The product was in the form of a yellow solid and was obtained with a yield of 89% (5.16 g).

### Example D

### Preparation of 2-fluoro-N-methylpyridinium tosylate from 2-chloro-N-methylpyridinium tosylate

In a 50 ml round-bottomed flask topped with a condenser, 2-chloro-Nmethylpyridinium tosylate (4.77 g, 15.9 mmol) was dissolved in 20 ml of acetonitrile.

Added to this solution was “spray dried” potassium fluoride (1.02 g, 17.5 mmol, 1.1 eq.) previously dried under a reduced pressure of around 20 mmHg at high temperature.

The whole mixture was refluxed for one hour.

The potassium chloride was filtered over a Büchner funnel after cooling the solution.

The filtrate was concentrated under a reduced pressure of around 20 mmHg, then was redissolved in 100 ml of dichloromethane.

The mixture was filtered again which made it possible to remove the excess potassium fluoride.

The filtrate was concentrated again under a reduced pressure of around 20 mmHg.

The solid recovered was then finely ground in methyl t-butyl ether for one hour then the mixture was filtered.

The product was in the form of a yellow solid and was obtained with a yield of 90%.

The NMR characteristics were the following:

¹H NMR (300 MHz, CDCl₃): 2.31 (s, 3H); 4.29 (d, J=3.8 Hz, 3H); 7.10 (d, J=8 Hz, 2H); 7.58 (d, J=8 Hz, 2H); 7.62 (dd, J=8.4 Hz, J=4.2 Hz, 1H); 7.79 (m, 1H); 8.52 (m, 1H); 9.07 (m, 1H).

¹³C NMR (75 MHz, CDCl₃)
Primaries: 21.3; 42.0 (d, J=5.3 Hz).
Secondaries: -
Tertiaries: 114.0 (d, J=19.9 Hz); 124.3 (d, J=3.8 Hz); 125.8 (2C); 128.8 (2C); 145.8 (d, J=7.7 Hz); 150.9 (d, J=11 Hz).
Quaternaries: 139.9; 142.6; 158.6 (d, J=278.3 Hz).

### Example E

### Preparation of 2-fluoro-N-methylpyridinium triflate from 2-fluoropyridine

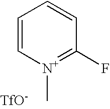

In a 25 ml round-bottomed flask, 2-fluoropyridine (2 g, 20.6 mmol) was diluted in 15 ml of toluene.

Using a syringe, methyl triflate (2.33 ml, 20.6 mmol) was added to this solution.

After a few minutes, a white precipitate was formed.

The mixture was left stirring magnetically at room temperature for one hour.

The precipitate was then filtered over a Büchner funnel.

The traces of solvent were removed via evaporation under a reduced pressure of around 20 mmHg.

The product was in the form of a white solid and was obtained with a yield of 99%.

### Example F

### Preparation of 2-fluoro-N-methylpyridinium triflate from 2-chloro-N-methylpyridinium triflate

In a 50 ml round-bottomed flask topped with a condenser, 2-chloro-N-methylpyridinium triflate (2.7 g, 10 mmol) was dissolved in 15 ml of acetonitrile.

Added to this solution was “spray dried” potassium fluoride (0.64 g, 11 mmol, 1.1 eq.) previously dried under a reduced pressure of around 20 mmHg at high temperature.

The whole mixture was refluxed for one hour.

The potassium chloride was filtered over a Büchner funnel after cooling the solution.

The filtrate was concentrated under a reduced pressure of around 20 mmHg, then was redissolved in 100 ml of dichloromethane.

The solid was filtered again and dried under a reduced pressure of 20 mmHg.

The product was in the form of a white solid and was obtained with a yield of 99%.

The NMR characteristics were the following:

¹H NMR (300 MHz, DMSO): 4.11 (d, J=4.1 Hz, 3H), 7.86 (m, 1H), 7.98 (dd, J=4.5 Hz, J=8 Hz), 8.62 (m, 1H), 8.80 (m, 1H).

¹³C NMR (75 MHz, DMSO):
Primaries: 41.9 (d, J=5.3 Hz).
Secondaries: -
Tertiaries: 114.6 (d, J=20.3 Hz); 124.2 (d, J=3.7 Hz); 144.9 (d, J=7.6 Hz); 151.2 (d, J=11.6 Hz).
Quaternaries: 157.8 (d, J=276.7 Hz).

### Example G

### Preparation of 2-fluoro-N-methylpyridinium fluoride from 2-fluoro-N-methylpyridinium triflate

In a 100 ml round-bottomed flask, 2-fluoro-N-methylpyridinium triflate (10 mmol) was dissolved in a minimum of acetonitrile (5 ml).

Added to this mixture was TBAT dissolved in 50 ml of dichloromethane.

A white precipitate formed immediately.

The latter was filtered over a Büchner funnel and washed with dichloromethane.

The solid was then dried under a reduced pressure of around 20 mmHg.

### Example H

### Preparation of 2-fluoro-N-methylpyridinium fluoride from 2-fluoro-N-methylpyridinium triflate

In a 25 ml round-bottomed flask, 2-fluoro-N-methylpyridinium tosylate (10 mmol) was dissolved in 10 ml of dichloromethane.

Added to this mixture was TBAT dissolved in 10 ml of dichloromethane.

A white precipitate formed immediately.

The latter was filtered over a Büchner funnel and washed with dichloromethane.

The solid was then to be dried under a reduced pressure of around 20 mmHg.

The ion exchange was quantitative regardless of the method.

The NMR characteristics were the following:

¹H NMR (300 MHz, DMSO): 4.11 (d, J=4.1 Hz, 3H), 7.86 (ddd, J=1.2 Hz, J=6.3 Hz, J=7.5 Hz, 1H), 7.99 (ddd, J=1 Hz, J=4.6 Hz, J=8.6 Hz), 8.62 (m, 1H), 8.81 (ddd, J=1.8 Hz, J=4.6 Hz, J=6.3 Hz, 1H).

¹³C NMR (75 MHz, DMSO):
Primaries: 41.6 (d, J=5 Hz).
Secondaries: -
Tertiaries: 114.3 (d, J=20.3 Hz); 123.9 (d, J=3.8 Hz); 144.8 Hz (d, J=7.6 Hz); 150.8 (d, J=11.6 Hz).
Quaternaries: 158.9 (d, J=271.9 Hz).

### Example I

### Preparation of N-methyl-2-chloroquinolinium triflate

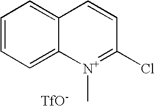

In a 50 ml round-bottomed flask, 2-chloroquinoline (20 mmol) was dissolved in 30 ml of toluene.

The mixture was cooled in an ice bath and methyl triflate (11 eq.) was added.

The whole mixture was left stirring for 8 hours at room temperature.

The white solid that precipitated was then filtered and washed with toluene.

It was then dried under a reduced pressure of around 20 mmHg.

The quinolinium salt was obtained with a yield of 95%.

The NMR characteristics were the following:

¹H NMR (CDCl₃, 300 MHz): 0.80 (s, 3H); 7.97 (m, 1H); 8.04 (d, J=8.8 Hz, 1H); 8.22-8.3 (m, 2H); 8.47 (d, J=9.5 Hz, 1H); 8.94 (d, J=8.8 Hz, 1H).

### Example J

### Preparation of N-methyl-2-fluoroquinolinium triflate

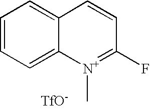

The same procedure was used as for obtaining N-methyl-2-fluoropyridinium triflate from N-methyl-2-chloropyrdinium triflate, with similar yields.

### Example K

### Preparation of N-methyl-2-fluoroquinolinium fluoride

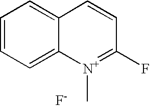

The same procedure was used as for obtaining N-methyl-2-fluoropyridinium fluoride from N-methyl-2-fluoropyridinium triflate, with similar yields.

### Example 1

### Preparation of 1-fluorodecane

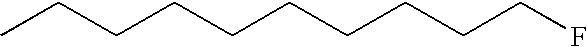

In a 5 ml round-bottomed flask, tetrabutylammonium hydrogendifluoride (560 mg, 2 mmol) was dried under a reduced pressure of 1 mmHg, at 100° C. for ½ hour.

After cooling, triethylamine (0.14 ml, 1 mmol) was added.

The whole mixture was dissolved in chloroform, then 1-decanol (158 mg, 1 mmol) and 1-methyl-2-fluoro-pyridinium tosylate (560 mg, 2 mmol) were added.

The mixture was heated under reflux of chloroform for 5 hours.

It was then hydrolyzed with 2 ml of water and neutralized with a saturated aqueous solution of sodium monohydrogencarbonate.

The extraction was carried out with 4 times 5 ml of petroleum ether.

The organic phase was dried over magnesium sulfate, filtered and concentrated under a reduced pressure of 250 mmHg.

The residue was purified by chromatography on a silica column (eluent: petroleum ether).

After evaporation, the product was then in the form of a transparent liquid and was obtained with a yield of 56% (m=90 mg).

The NMR characteristics were the following:

¹H NMR (CDCl₃, 300 MHz): 0.81 (t, J=8 Hz, 3H); 1.1-1.3 (m, 14H); 1.5-1.7 (m, 2H); 4.37 (dt, J=47.4 Hz, J=6.2 Hz, 2H).

¹³C NMR (CDCl₃, 75 MHz):
Primaries: 14.1.
Secondaries: 22.7; 25.2; 25.3; 29.3 (d, J=4 Hz); 29.5; 30.4 (d, J=19 Hz); 31.9; 84.3 (d, J=164 Hz).
Tertiaries: -
Quaternaries: -

### Example 2

### Preparation of 2-fluorodecane

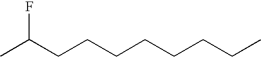

In a 5 ml round-bottomed flask, tetrabutylammonium hydrogendifluoride (560 mg, 2 mmol) was dried under a reduced pressure of 1 mmHg, at 100° C. for ½ hour.

After cooling, triethylamine (0.14 ml, 1 mmol) was added.

The whole mixture was dissolved in chloroform, then 2-decanol (158 mg, 1 mmol and 1-methyl-2-fluoro-pyridinium tosylate (560 mg, 2 mmol) were added.

The mixture was heated under reflux of chloroform for 5 hours.

It was then hydrolyzed with 2 ml of water and neutralized with a saturated aqueous solution of sodium monohydrogencarbonate.

The extraction was carried out with 4 times 5 ml of petroleum ether.

The organic phase was dried over magnesium sulfate, filtered and concentrated under a reduced pressure of 250 mmHg.

The residue was purified by chromatography on a silica column (eluent: petroleum ether).

The product was then in the form of a transparent liquid and was obtained with a yield of 43% (m=69 mg).

The NMR characteristics were the following:

¹H NMR (CDCl₃, 300 MHz): 0.75-0.85 (m, 6H); 1.1-1.3 (m, 14H); 4.37 (m, 1H).

¹³C NMR (CDCl₃, 75 MHz):
Primaries: 14.1; 21.0 (d, J=23 Hz).
Secondaries: 22.3; 22.6; 25.1 (d, J=5 Hz); 29.2; 29.5 (d, J=2 Hz); 31.9; 37.0 (d, J=21 Hz).
Tertiaries: 91.1 (d, J=164 Hz).

### Quaternaries: -

### Example 3

### Preparation of 2-fluoro-1,2-diphenylethanone

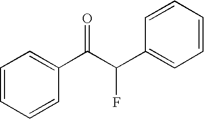

In a 5 ml round-bottomed flask, tetrabutylammonium hydrogendifluoride (280 mg, 1 mmol) was dried under a reduced pressure of 1 mmHg at 100° C. for ½ hour.

After cooling, triethylamine (0.07 ml, 1 mmol) was added.

The whole mixture was dissolved in chloroform, then benzoin (106 mg, 0.5 mmol) and 1-methyl-2-fluoro-pyridinium tosylate (280 mg, 1 mmol) were added.

The mixture was heated under reflux of chloroform overnight.

It was then hydrolyzed with 2 ml of water and neutralized with a saturated aqueous solution of sodium monohydrogencarbonate.

The extraction was carried out with 4 times 5 ml of ethyl ether.

The organic phase was dried over magnesium sulfate, filtered and concentrated under a reduced pressure of 20 mmHg.

The residue was purified by chromatography on a silica column (eluent: petroleum ether/dichloromethane:1/1; R_{f}=0.25).

The product was then in the form of a white solid (melting point: 53° C.) and was obtained with a yield of 87% (m=93 mg).

The NMR characteristics were the following:

¹H NMR (CDCl₃, 300 MHz): 6.52 (d, J=48.7 Hz, 1H); 7.3-7.6 (m, 8H); 7.9-8.0 (m, 2H).

¹³C NMR (CDCl₃, 75 MHz):
Primaries: -
Secondaries: -
Tertiaries: 94.0 (d, J=186 Hz); 127.3 (d, J=6 Hz); 128.7; 129.1; 129.1; 129.6 (d, J=3 Hz) 133.8.
Quaternaries: 134.1; 134.3 (d, J=20 Hz); 194.3 (d, J=21 Hz).

### Example 4

### Preparation of ethyl fluorophenylacetate

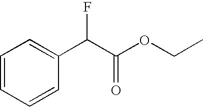

In a 5 ml round-bottomed flask, tetrabutylammonium hydrogendifluoride (280 mg, 1 mmol) was dried under a reduced pressure of 1 mmHg at 100° C. for ½ hour.

After cooling, triethylamine (0.07 ml, 1 mmol) was added.

The whole mixture was dissolved in chloroform (1 ml), then ethyl mandelate (90 mg, 0.5 mmol) and 1-methyl-2-fluoropyridinium tosylate (280 mg, 1 mmol) were added.

The mixture was heated under a reflux of chloroform for three hours.

It was then hydrolyzed with 5 ml of water.

The extraction was carried out with 3 times 5 ml of ethyl ether.

The organic phase was dried over magnesium sulfate, filtered and concentrated under a reduced pressure of around 20 mmHg.

The residue was purified by chromatography on a silica column (eluent: petroleum ether/dichloromethane:1/1).

The product was then in the form of a colorless liquid and was obtained with a yield of 56% (m=51 mg).

The NMR characteristics were the following:

¹H NMR (CDCl₃ 300 MHz): 1.29 (t, J=7.3 Hz; 3H) 4.25 (q, J=7.3 Hz; 2H); 5.76 (d, J=48.2 Hz; 1H); 7.10-7.48 (m, 5H).

### Example 5

### Preparation of isopropyl fluorophenylacetate

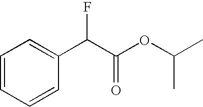

In a 5 ml round-bottomed flask, tetrabutylammonium hydrogendifluoride (280 mg, 1 mmol) was dried under a reduced pressure of 1 mmHg at 100° C. for ½ hour.

After cooling, triethylamine (0.07 ml, 1 mmol) was added.

The whole mixture was dissolved in chloroform (1 ml), then isopropyl mandelate (90 mg, 0.5 mmol) and 1-methyl-2-fluoropyridinium tosylate (280 mg, 1 mmol) were added.

The mixture was heated under a reflux of chloroform for three hours.

It was then hydrolyzed with 5 ml of water.

The extraction was carried out with 3 times 5 ml of ethyl ether.

The organic phase was dried over magnesium sulfate, filtered and concentrated under a reduced pressure of around 20 mmHg.

The residue was purified by chromatography on a silica column (eluent: petroleum ether/dichloromethane:1/1).

The product was then in the form of a colorless liquid and was obtained with a yield of 63% (m=62 mg).

The NMR characteristics were the following:

¹H NMR (CDCl₃, 300 MHz): 1.20 (t, d=6.3 Hz; 3H); 1.30 (t, d=6.3 Hz; 3H); 5.12 (spt, J=6.3 Hz; 1H); 5.76 (d, J=48.0 Hz; 1H) 7.10-7.48 (m, 5H).

¹³C NMR (CDCl₃, 75 MHz)
Primaries: 21.5; 21.7.
Secondaries: -
Tertiaries: 69.7; 89.4 (d, J=185 Hz); 126.6; 127.9; 128.7.
Quaternaries: 134.6 (d, J=38 Hz); 168.1 (d, J=27 Hz).

### Example 6

### Preparation of 1-difluoromethyl-4-methoxybenzene

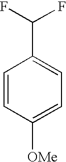

In a 25 ml round-bottomed flask tetrabutylammonium hydrogendifluoride monohydrate (3 g; 10 mmol; 3.3 eq.) was introduced.

The latter was heated at 100° C. in an oil bath under a reduced pressure of 1 mmHg for one hour.

After cooling under argon, 1-methyl-2-fluoropyridinium tosylate (2.8 g; 10 mmol; 3.3 eq.) was introduced followed by anisaldehyde (408 mg; 3 mmol) and triethylamine (1.4 ml; 10 mmol; 3.3 eq.).

After stirring for 5 minutes, the mixture was then brought to 80° C. and became completely homogeneous.

After 5 hours, the mixture was hydrolyzed with water (5 ml), and neutralized with a saturated solution of sodium hydrogencarbonate (10 ml).

The aqueous solution was then extracted with diethyl ether (3 times 20 ml).

The organic phase was dried over magnesium sulfate.

After filtration, the solvent was evaporated under a reduced pressure of around 20 mmHg.

The black liquid residue had, in thin-layer chromatography, two spots at respective Rf values 0.27 and 0.71 (petroleum ether/dichloromethane 1/1) or 0.08 and 0.41 (petroleum ether/dichloromethane 3/1).

Chromatography is carried out on a silica column by eluting with a petroleum ether/dichloromethane gradient of 3/1 to 1/1.

The 1-difluoromethyl-4-methoxybenzene was in the form of a slightly yellow oil (278 mg; 1.76 mmol; 59%).

The anisaldehyde recovered was a white solid (100 mg; 0.73 mmol; 24%).

The NMR characteristics were the following:

¹H NMR (CDCl₃, 300 MHz): 3.85 (s, 3H); 6.62 (t, J=56.8 Hz, 1H); 6.96 (d, J=8.9 Hz, 2H); 7.45 (d, J=8.9 Hz, 2H).

¹³C NMR (CDCl₃, 75 MHz):
Primaries: 55.34.
Secondaries: -
Tertiaries: 114.0; 114.9 (t, J=237 Hz); 127.1 (t, J=6 Hz).
Quaternaries: 126.5 (t, J=23 Hz), 161.4.

### Example 7

### Preparation of 1,4-bis(trifluoromethyl)benzene

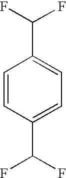

In a 5 ml round-bottomed flask, tetrabutylammonium hydrogendifluoride (750 mg, 2.5 mmol) was dried under a reduced pressure of 1 mmHg at 100° C. for 1 hour.

After cooling, triethylamine (0.35 ml, 2.5 mmol), 1-methyl-2-fluoropyridinium tosylate (700 mg, 2.5 mmol), then terephthaldehyde (36 mg, 0.25 mmol) were added.

The whole mixture was heated at 80° C. for 6 hours.

It was then hydrolyzed with 3 ml of water and neutralized with a saturated aqueous solution of sodium monohydrogencarbonate (3 ml).

The extraction was carried out with 3 times 5 ml of ethyl ether.

The organic phase was dried over magnesium sulfate, filtered and concentrated under a reduced pressure of around 20 mmHg.

The residue was purified by chromatography on a silica column (eluent: gradient of dichloromethane in petroleum ether).

The product was then in the form of a colorless liquid and was obtained with a yield of 30% (m=13 mg).

4-Difluoromethylbenzaldehyde was isolated with a yield of 20% (8 mg).

The chromatography results were:

Eluent: petroleum ether/dichloromethane:1/1.

Developer: UV.

Retardation factor: Rf₁=0.8; and
Rf₂=0.27.

The NMR characteristics were the following:

¹H NMR (CDCl₃, 300 MHz): 6.70 (t, J=56.5 Hz, 2H); 7.62 (s, 4H).

¹³C NMR (CDCl₃, 75 MHz):
Primaries: -
Secondaries: -
Tertiaries: 114.0 (t, J=239 Hz); 126.0 (t, J=6 Hz).
Quaternaries: 136.7 (t, J=22 Hz).

### 4-Difluoromethylbenzaldehyde

¹H NMR (CDCl₃ 300 MHz): 6.71 (t, J=55.9 Hz, 1H); 7.70 (d, J=7.9 Hz, 2H); 7.99 (d, J=7.9 Hz, 2H); 10.09 (s, 1H).

### Example 8

### Preparation of 10,10-difluorophenanthren-9-one

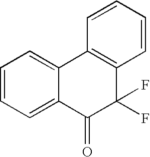

In a 10 ml round-bottomed flask, tetrabutylammonium hydrogendifluoride (2.8 g, 10 mmol) was dried under a reduced pressure of 1 mmHg at 100° C. for 1 hour.

After cooling, triethylamine (0.7 ml, 10 mmol) and 1-methyl-2-fluoropyridinium tosylate (2.8 g, 10 mmol) were added.

The whole mixture was left stirring magnetically until a homogeneous solution was obtained (slight heating may be necessary).

Phenanthrene-9,10-dione (208 mg, 1 mmol) was then added and the mixture was heated at 80° C. overnight.

It was then hydrolyzed with 3 ml of water and neutralized with a saturated aqueous solution of sodium monohydrogencarbonate.

The extraction was carried out with 4 times 10 ml of ether ethyl.

The organic phase was dried over magnesium sulfate, filtered and concentrated under a reduced pressure of around 20 mmHg.

The residue was purified by chromatography on a silica column (eluent: petroleum ether/dichloromethane:1/1; Rf=0.3).

The product was then in the form of a white solid (melting point: 90° C.) and was obtained with a yield of 58% (m=124 mg).

The NMR characteristics were the following:

¹H NMR (CDCl₃, 300 MHz): 7.48 (m, 2H); 7.61 (tq, J=1.3 Hz, J=7.5 Hz, 1H); 7.74 (ddd, J=1.5 Hz J=7.5 Hz J=8 Hz, 1H); 7.87 (dd, J=1 Hz, J=7.7 Hz, 1H); 7.94 (m, 2H); 8.09 (ddd, J=0.5 Hz, J=1.5 Hz, J=7.7 Hz, 1H).

¹³C NMR (CDCl₃, 75 MHz):
Primaries: -
Secondaries: -
Tertiaries: 123.7; 124.4; 127.3 (t, J=5 Hz); 128.8 (t, J=1 Hz); 129.3; 129.6 (t, J=1 Hz); 132.4 (t, J=2 Hz); 136.2.
Quaternaries: 108.0 (t, J=245 Hz); 127.7 (t, J=2 Hz); 130.2 (t, J=23 Hz); 131.7 (t, J=6 Hz); 136.1 (t, J=2 Hz); 186.9 (t, J=26 Hz).

1.-16. (canceled)

17. A process for the preparation of a monofluoro or difluoro hydrocarbon-based compound, which comprises reacting an alcohol or a carbonyl-based compound with a fluorinating reagent, optionally in the presence of a base, said fluorinating agent comprising a pyridinium reactant having the following formula: 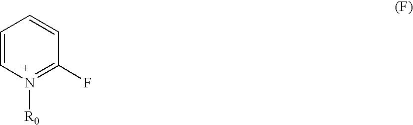
wherein:
R₀ is an alkyl or cycloalkyl radical.

18. The process as defined by claim 17, said fluorinating reagent having been prepared in situ by reacting a fluoride source with a halogenated pyridinium reactant having the following formula: 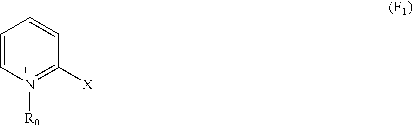
in which:
X is a halogen atom with a higher ranking and other than fluorine; and
R₀ is an alkyl or cycloalkyl radical.

19. The process as defined by claim 17 or 18, said fluorinating reagent having the formula (F) or (F₁) being included in a polycyclic structure, the pyridinium ring being fused to a saturated, unsaturated or aromatic ring having 5 or 6 carbon atoms.

20. The process as defined by claim 19, said fluorinating reagent having the formula (F) or (F₁) in which R₀ is a C₁-C₄ alkyl radical.

21. The process as defined by claims 17 or 18, said fluorinating reagent comprising a pyridinium compound in which the quaternized nitrogen atom is associated with a Y⁻ counterion selected from among halides, or sulfonate or carboxylate groups.

22. The process as defined by claim 21, said fluorinating reagent being selected from the group consisting of:
2-fluoro-N-methylpyridinium tosylate;
2-fluoro-N-methylpyridinium triflate;
2-fluoro-N-methylpyridinium fluoride;
N-methyl-2-fluoroquinolinium triflate; and
N-methyl-2-fluoroquinolinium fluoride.

23. The process as defined by claim 17, comprising reacting an alcohol having the general formula (I):
R₁—OH (I)
wherein:
R₁ is a hydrocarbon-based radical having from 1 to 30 carbon atoms, which may be a linear or branched, saturated or unsaturated acyclic aliphatic radical; a saturated, unsaturated or aromatic cycloaliphatic radical; a linear or branched, saturated or unsaturated aliphatic radical bearing a cyclic substituent.

24. The process as defined by claim 17, comprising reacting a carbonyl-based aldehyde or ketone (or diketone) having one of the general formulae: 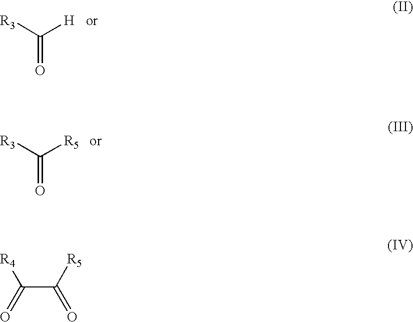
wherein:
R₃, R₄ and R₅, which may be identical or different, are each a hydrocarbon-based radical having from 1 to 40 carbon atoms, which may be a linear or branched, saturated or unsaturated acyclic aliphatic radical; a monocyclic or polycyclic, saturated, unsaturated or aromatic carbocyclic or heterocyclic radical; or a chain of the aforementioned radicals; with the proviso that R₄ and R₅ may together form a ring member having 5 or 6 atoms; and the R₄ and R₅ radicals do not contain hydrogen atoms on the carbon atom in position α with respect to the carbonyl group.

25. The process as defined by claim 17, wherein the ratio between the number of moles of fluorinating reagent and the number of moles of coreactant ranges from 1 to 3.

26. The process as defined by claim 17, carried out in the presence of a base, the pKa of which is at least greater than or equal to 4.

27. The process as defined by claim 26, said base comprising a carbonate, hydrogencarbonate, phosphate, or hydrogenphosphate of an alkaline metal, or of an alkaline-earth metal, or an organic base.

28. The process as defined by claim 18, said fluoride source comprising hydrofluoric acid; a salt; or a quaternary ammonium fluoride.

29. The process as defined by claim 17, carried out in the presence of an organic solvent selected from the group consisting of dimethyl sulfoxide, sulfolane and linear or cyclic carboxamides, N,N-dimethylacetamide (DMAC), N,N-diethylacetamide, dimethylformamide (DMF) and diethylformamide; aliphatic and aromatic nitrites, acetonitrile; halogenated and non-halogenated aliphatic, cycloaliphatic or aromatic hydrocarbons; and ethers.

30. The process as defined by claim 17, wherein the fluorination reaction is carried out at a temperature ranging from 0° C. to 140° C.

31. The process as defined by claim 17, comprising preparing a monofluoro compound from an alcohol.

32. The process as defined by claim 17, comprising preparing a gem-difluoro compound from a carbonyl-based compound.

33. The process as defined by claim 25, said ratio ranging from 1.5 to 2.

34. The process as defined by claim 26, said pKa ranging from 7 to 11.

35. The process as defined by claim 30, carried out at a temperature ranging from 80° to 100°.
